(19) United States
(12) Patent Application Publication
(10) Pub. No.: US 2004/0202635 A1
(43) Pub. Date: Oct. 14, 2004

(54) Cosmetic cleansing formulations based on a combination of sodium laureth sulfate, alkylpolyamphopolycarboxyglycinates and N-acylamino acid salts

(75) Inventors: Andreas Clausen, Hamburg (DE); Ursula Jensen, Hamburg (DE); Stephan Ruppert, Hamburg (DE); Martin Sugar, Hamburg (DE); Maren Wilken, Norderstedt (DE)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Beiersdorf AG

(21) Appl. No.: 10/770,096

(22) Filed: Feb. 2, 2004

Related U.S. Application Data
(63) Continuation of application No. PCT/EP02/08502, filed on Jul. 31, 2002.

(30) Foreign Application Priority Data
Aug. 2, 2001 (DE) 10138035.6
Oct. 11, 2001 (DE) 10150409.8

(51) Int. Cl. A61K 7/075; A61K 7/08

(52) U.S. Cl. 424/70.22; 424/70.24

(57) ABSTRACT

The present invention is a cosmetic cleansing formulation that includes sulfates or sulfonates, in particular sodium laureth sulfate, alkylpolyamphopolycarboxyglycinates and N-acylamino acid salts.

### CROSS-REFERENCE TO RELATED APPLICATIONS

This is a continuation application of PCT/EP02/08502, filed Jul. 31, 2002, which is incorporated herein by reference in its entirety, and also claims the benefit of German Priority Application No. 101 38 035.6, filed Aug. 2, 2001 and German Priority Application No. 101 50 409.8, filed Oct. 11, 2001.

### FIELD OF THE INVENTION

The present invention relates to the use of a surfactant combination of sulfates or sulfonates, in particular sodium laureth sulfate, alkylpolyamphopoly-carboxyglycinates and N-acylamino acid salts in cosmetic cleansers.

### BACKGROUND OF THE INVENTION

Surfactants are of major importance as washing-active substances in cosmetic cleansers. As a result of their specific molecular structure having in each case one hydrophilic (water-attracting) and hydrophobic (water-repelling) group in the same molecule, they are able to reduce the surface tension of water, to wet the skin, to facilitate the removal of dirt and to regulate foam.

A distinction is made between four classes of surfactants:

A first class is the nonionic surfactants. These include fatty alcohol ethoxylates [RO(CH₂CH₂O)ₙH], fatty acid monoethanolamides [RCONHCH₂CH₂OH] and alkyl polyglycosides (APGs)

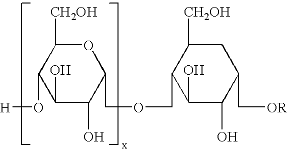

R=fatty acid radical.

A second class is the amphoteric surfactants. These are compounds which contain both a cationic function, in most cases a quaternary nitrogen, and an anionic function, in most cases a carboxylate group. Their charge state is pH-dependent. They include alkylaminobetaines

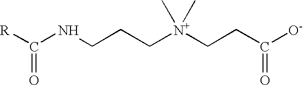

and also alkylamidoglycinates

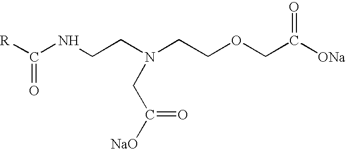

R=fatty acid radical

The group of cationic surfactants consists of compounds which contain at least one quaternary nitrogen atom. These include, for example, alkylamines, alkylimidazoles, and ethoxylated amines.

The group of anionic surfactants is formed from sulfates, sulfonates and carboxylates, i.e., salts of esters of sulfuric acid, and also salts of sulfonic and carboxylic acids.

The most common surfactants from this class are derived from lauryl alcohol (C₁₂H₂₅OH). These are the sodium salt of lauryl sulfate (sodium lauryl sulfate, SLS) with the structure C₁₂H₂₅OSO₃ and ethylene glycol ether of the alcohol, which are likewise esterified with sulfuric acid (sodium laureth sulfate, SLES). These have the structure:

C₁₂H₂₅ (OCH₂CH₂)ₙ OSO₃.

Standard commercial sodium lauryl ether sulfate (sodium polyoxyethylene lauryl sulfate) which has been used in the present invention is, however, usually a mixture of substances whose structures obey the general formula

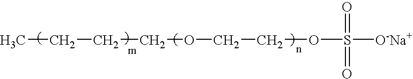

where m can assume the numbers 4 to 6 and n can assume the numbers 0 to 10.

These compounds have excellent washing-active properties and good foaming ability. However, in relatively high concentrations, they have an irritative effect on skin and mucosa. In addition, they lead to a reduction in skin moisture and to an increase in the transepidermal water loss (TEWL). Due to the good availability, the excellent washing properties and last but not least due to the acceptable cost, it is barely possible to dispense completely with this surfactant.

### SUMMARY OF THE INVENTION

It was therefore the object of the present invention to develop more skin-friendly cosmetic cleansing formulations based on sodium laureth sulfate.

Surprisingly, it was possible to overcome the shortcomings of the prior art using a mixture of the surfactants sodium laureth sulfate, one or more alkylpolyamphopolycarboxyglycinates and one or more N-acylamino acid salts, and to formulate cosmetic cleansers with good cleansing performance and high mildness. The cosmetic formulations according to the invention are characterized by a pleasant appearance and sensory properties, pleasant caring feel on the skin and a superior foam formation. Surprisingly, only small amounts of co-surfactants are required in order to achieve this effect. Though U.S. Pat. No. 5,693,604 and U.S. Pat. No. 5,387,372 mention surfactant combinations containing sodium laureth sulfate and N-alkylamino acid salts, in these cases amphocarboxyglycinate is used instead of the polyglycinate. Moreover, mixtures of five different surfactants are used which differ significantly in terms of their composition from the surfactant mixture according to the invention and therefore offered just as little to pave the way to the invention, as did WO 9959531 (combination of amphocarboxypolyglycinates and silicones), WO 9511004 (combination of amphocarboxypolyglycinates and alkoxylated amide) and WO 9501153 (combination of amphocarboxypolyglycinates and 2 monoethanolamides).

### DETAILED DESCRIPTION OF THE PREFERRED EMBODIMENTS

According to the invention, the use of sodium carboxymethylcocoylpoly-propylamine as an alkylpolyamphopolycarboxyglycinate (INCI: sodium carboxy-methyl coco polypropylamine, e.g.: Ampholak 7CX/C [Akzo Nobel]) is advantageous, as is sodium cocoyl glutamate (INCI: sodium cocoyl glutamate) as an N-acylamino acid salt, which is used according to the invention advantageously in the form of its disodium salt (INCI: disodium cocoyl glutamate).

According to the invention, it is advantageous to choose the following concentrations for the individual surfactants:

Sodium laureth sulfate: 0.1 to 30% by weight, in particular 9 to 15% by weight and very particularly advantageously 10% by weight, based on the total weight of the formulation.

Alkylpolyamphopolycarboxyglycinates: 0.1 to 10% by weight, in particular 0.1 to 5% by weight and very particularly preferably 2% by weight, based on the total weight of the formulation.

N-Acylamino acids: 0.1 to 10% by weight, in particular 0.5 to 5% by weight and very particularly preferably 2% by weight, based on the total weight of the formulation.

In addition, the cosmetic cleansing formulations can, in accordance with the invention, advantageously comprise:

anionic surfactants

acylamino acids (and salts thereof), such as

1. acyl glutamates, for example sodium acyl glutamate, di-TEA-palmitoyl aspartate and sodium caprylic/capric glutamate,

2. acylpeptides, for example palmitoyl-hydrolyzed milk protein, sodium cocoyl-hydrolyzed soya protein and sodium/potassium cocoyl-hydrolyzed collagen,

3. sarcosinates, for example myristoyl sarcosine, TEA-lauroyl sarcosinate, sodium lauroyl sarcosinate and sodium cocoyl sarcosinate,

4. taurates, for example sodium lauroyl taurate and sodium methylcocoyl taurate,

5. acyl lactylates, lauroyl lactylate, caproyl lactylate, and

6. alaninates,

carboxylic acids and derivatives, such as

1. carboxylic acids, for example lauric acid, aluminium stearate, magnesium alkanolate and zinc undecylenate,

2. ester carboxylic acids, for example calcium stearoyl lactylate, laureth-6 citrate and sodium PEG-4 lauramide carboxylate, and

3. ether carboxylic acids, for example sodium laureth-13 carboxylate and sodium PEG-6 cocamide carboxylate,

phosphoric esters and salts, such as, for example, DEA-oleth-10 phosphate and dilaureth4 phosphate,

sulfonic acids and salts, such as

1. acyl isethionates, e.g. sodium/ammonium cocoyl isethionate,

2. alkylarylsulfonates,

3. alkylsulfonates, for example sodium cocomonoglyceride sulfate, sodium C₁₂-₁₄-olefinsulfonate, sodium lauryl sulfoacetate and magnesium PEG-3 cocamide sulfate, and

4. sulfosuccinates, for example dioctyl sodium sulfosuccinate, disodium laureth sulfosuccinate, disodium lauryl sulfosuccinate and disodium undecyleneamido-MEA sulfosuccinate and

sulfuric esters, such as

1. alkyl ether sulfate, for example sodium, ammonium, magnesium, MIPA, TIPA laureth sulfate, sodium myreth sulfate and sodium C₁₂₋₁₃ pareth sulfate, and

2. alkyl sulfates, for example sodium, ammonium and TEA lauryl sulfate.

B. Cationic Surfactants

Cationic surfactants which can optionally be used advantageously are

1. alkylamines,

2. alkylimidazoles,

3. ethoxylated amines and

4. quaternary surfactants, and

5. ester quats.

Advantageous quaternary surfactants are alkylbetaine, alkylamidopropylbetaine and alkylamidopropylhydroxysulfane.

Cationic surfactants can also preferably be chosen for the purposes of the present invention from the group of quaternary ammonium compounds, in particular benzyltrialkylammonium chlorides or bromides, such as, for example, benzyldimethylstearylammonium chloride, and also alkyltrialkylammonium salts, for example cetyltrimethylammonium chloride or bromide, alkyldimethylhydroxyethylammonium chlorides or bromides, dialkyldimethyl-ammonium chlorides or bromides, alkylamidoethyltrimethylammonium ether sulfates, alkylpyridinium salts, for example lauryl- or cetylpyrimidinium chloride, imidazoline derivatives and compounds having cationic character, such as amine oxides, for example alkyldimethylamine oxides or alkylaminoethyl-dimethylamine oxides. In particular the use of cetyltrimethylammonium salts is advantageous.

C. Amphoteric Surfactants

Amphoteric surfactants which can be used advantageously are

1. acyl/dialkylethylenediamine, for example sodium acyl amphoacetate, disodium acyl amphodipropionate, disodium alkyl amphodiacetate, sodium acyl amphohydroxypropylsulfonate, disodium acyl amphodiacetate and sodium acyl amphopropionate, and

2. N-alkylamino acids, for example aminopropylalkylglutamide, alkylaminopropionic acid, sodium alkylimidodipropionate and lauroamphocarboxyglycinate.

D. Nonionic surfactants

Nonionic surfactants which can be used advantageously are

1. alcohols,

2. alkanolamides, such as cocamides MEA/DEA/MIPA,

3. amine oxides, such as cocoamidopropylamine oxide,

4. esters which are formed by esterification of carboxylic acids with ethylene oxide, glycerol, sorbitan or other alcohols,

5. ethers, for example ethoxylated/propoxylated alcohols, ethoxylated/propoxylated esters, ethoxylated/propoxylated glycerol esters, ethoxylated/propoxylated cholesterols, ethoxylated/propoxylated triglyceride esters, ethoxylated propoxylated lanolin, ethoxylated/propoxylated polysiloxanes, propoxylated POE ethers and alkyl polyglycosides, such as lauryl glucoside, decyl glycoside and cocoglycoside,

6. sucrose esters, sucrose ethers

7. polyglycerol esters, diglycerol esters, monoglycerol esters, and

8. methylglucose esters, esters of hydroxy acids.

Cleansing preparations according to the invention are advantageously in the form of gels and comprise one or more gel formers or hydrocolloids.

“Hydrocolloid” is the technological abbreviation for the more correct name “hydrophilic colloid”. Hydrocolloids are macromolecules which have a largely linear structure and have intermolecular forces of interaction which permit secondary and primary valence bonds between the individual molecules and thus the formation of a recticular structure. Some are water-soluble natural or synthetic polymers which, in aqueous systems, form gels or viscous solutions. They increase the viscosity of the water by either binding water molecules (hydration) or else by absorbing and encapsulating the water into their interwoven macromolecules, at the same time as restricting the mobility of the water. Such water-soluble polymers represent a large group of chemically very different natural and synthetic polymers whose common feature is their solubility in water or aqueous media. A prerequisite for this is that these polymers have a number of hydrophilic groups sufficient for solubility in water and are not too greatly crosslinked. The hydrophilic groups may be nonionic, anionic or cationic in nature, for example as follows:

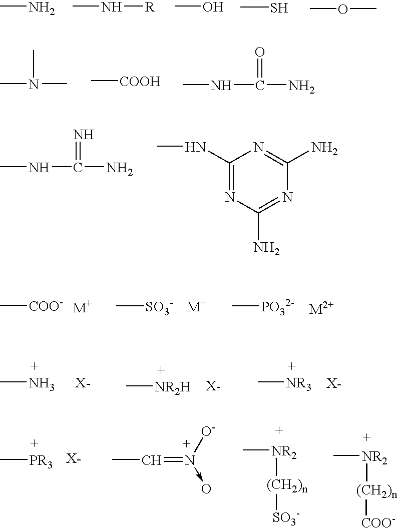

The group of the cosmetically and dermatologically relevant hydrocolloids can be divided as follows into:

organic, natural compounds, such as, for example, agar agar, carrageen, tragacanth, gum arabic, alginates, pectins, polyoses, guar flour, carob bean flour, starch, dextrins, gelatins, caseine,

organic, modified natural substances, such as, for example, carboxymethylcellulose and other cellulose ethers, hydroxyethylcellulose and hydroxypropylcellulose and the like,

organic, completely synthetic compounds, such as, for example, polyacrylic- and polymethacrylic compounds, vinyl polymers, polycarboxylic acids, polyethers, polyimines, polyamides, and

inorganic compounds, such as, for example, polysilicic acids, clay minerals, such as montmorillonites, zeolites, and silicas.

Hydrocolloids which are preferred according to the invention are for example, methylcelluloses, which is the term used for the methyl ethers of cellulose. They are characterized by the following structural formula

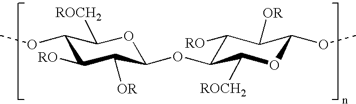

in which R may be a hydrogen or a methyl group.

Particularly advantageous for the purposes of the present invention are the cellulose mixed ethers, which are generally likewise referred to as methylcelluloses, which contain, in addition to a predominating content of methyl groups, additionally 2-hydroxyethyl groups, 2-hydroxypropyl groups or 2-hydroxybutyl groups. Particular preference is given to (hydroxypropyl)methylcelluloses, for example those available under the trade name Methocel® E4M from Dow Chemical Comp.

Also advantageous according to the invention is sodium carboxymethylcellulose, the sodium salt of the glycolic ether of cellulose, for which R in structural formula I may be a hydrogen and/or CH₂—COONa. Preference is given to the sodium carboxymethylcellulose available under the trade name Natrosol Plus 330CS from Aqualon, also referred to as cellulose guru.

Also preferred for the purposes of the present invention is xanthan (CAS No. 11138-66-2), also called xanthan gum, which is an anionic heteropolysaccharide which is usually formed by fermentation from corn sugar and is isolated as the potassium salt. It is produced by Xanthomonas campestris and some other species under aerobic conditions and has a molecular weight of from 2×10⁶ to 24×10⁶. Xanthan is formed from a chain having β-1,4-bonded glucose (cellulose) with side chains. The structure of the subgroups consists of glucose, mannose, glucuronic acid, acetate and pyruvate. Xanthan is the name given to the first microbial anionic heteropolysaccharide. It is produced by Xanthomonas campestris and some other species under aerobic conditions and has a molecular weight of from 2-15 10⁶. Xanthan is formed from a chain having β-1,4-bonded glucose (cellulose) with side chains. The structure of the subgroups consists of glucose, mannose, glucuronic acid, acetate and pyruvate. The number of pyruvate units determines the viscosity of the xanthan. Xanthan is produced in two-day batch cultures with a yield of 70-90%, based on carbohydrate used. In this connection, yields of 25-30 g/l are achieved. After the culture has been destroyed, work-up takes place by precipitation with, for example, 2-propanol. Xanthan is then dried and ground.

An advantageous gel former for the purposes of the present invention is also carrageen, a gel-forming extract with a similar structure to agar, from North Atlantic red algae, which belong to the Florideae (Chondrus crispus and Gigartina stellata).

The term carrageen is frequently used for the dried algae product and carrageenan for the extract thereof. The carrageen precipitated from the hot-water extract of the algae is a colorless to sand-colored powder with a molecular weight range from 100 000-800 000 and a sulfate content of about 25%. Carrageen, which is very readily soluble in warm water, forms a thixotropic gel upon cooling, even if the water content is 95-98%. The rigidity of the gel is effected by the double helix structure of carrageen. In the case of carrageenan, three main constituents are differentiated: the gel-forming κ fraction consists of D-galactose 4-sulfate and 3,6-anhydro-α-D-galactose, which has alternate glycoside bonds in the 1,3- and 1,4-position (by contrast, agar contains 3,6-anhydro-α-L-galactose). The nongelling λ fraction is composed of 1,3-glycosidically linked D-galactose 2-sulfate and 1,4-bonded D-galactose-2,6-disulfate radicals, and is readily soluble in cold water. The [symbol] -Carrageenan, composed of D-galactose 4-sulfate in 1,3 bond and 3,6-anhydro-α-D-galactose 2-sulfate in 1,4 bond, is both water-soluble and also gel-forming. Further carrageen grades are likewise referred to using Greek letters. The type of cations present (K⁺, NH₄⁺, Na⁺, Mg²⁺, Ca²⁺) also influences the solubility of the carrageens.

Polyacrylates are gelling agents likewise to be used advantageously for the purposes of the present invention. Polyacrylates advantageous according to the invention are acrylate-alkyl acrylate copolymers, in particular those chosen from the group of so-called carbomers or carbopols (Carbopol® is actually a registered trademark of NOVEON Inc.). In particular, the acrylate-alkyl acrylate copolymers advantageous according to the invention are characterized by the following structure:

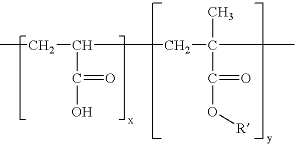

where R′ is a long-chain alkyl radical, and x and y represent numbers which symbolize the respective stoichiometric proportion of each of the comonomers.

According to the invention, preference is given to acrylate copolymers and/or acrylate-alkyl acrylate copolymers which are available under the trade names Carbopol® 1382, Carbopol® 981 and Carbopol® 5984, Aqua SF-1 from NOVEON Inc. and as Aculyn® 33 from International Specialty Products Corp.

Also advantageous are copolymers of C₁₀₋₃₀-alkyl acrylates and one or more monomers of acrylic acid, of methacrylic acid or esters thereof which are crosslinked with an allyl ether of sucrose or an allyl ether of pentaerythritol.

Compounds which carry the INCI name “Acrylates/C₁₀₋₃₀ Alkyl Acrylate Crosspolymer” are advantageous. Particularly advantageous are those polymers available under the trade names Pemulen TR1 and Pemulen TR2 from NOVEON Inc.

Also advantageous are compounds which the INCI name “acrylates/C12-24 pareth-25 acrylate copolymer” (obtainable under the trade names Synthalen® W2000 from 3V Inc.), which the INCI name “acrylates/steareth-20 methacrylate copolymer” (obtainable under the trade names Aculyn® 22 from International Specialty Products Corp.), which the INCI name “acrylates/steareth-20 itaconate copolymer” (obtainable under the trade names Structure 2001® from National Starch), which the INCI name “acrylates/aminoacrylates/C10-30 alkyl PEG-20 itaconate copolymer” (obtainable under the trade names Structure Plus® from National Starch) and similar polymers.

The total amount of one or more hydrocolloids in the finished cosmetic or dermatological preparations is advantageously chosen to be less than 1.5% by weight, preferably between 0.1 and 1.0% by weight, based on the total weight of the preparations.

It is advantageous for the purposes of the present invention if the content of one or more polyacrylates in the cosmetic or dermatological cleansing emulsion is chosen from the range from 0.5 to 4% by weight, very particularly advantageously from 0.7 to 2% by weight, in each case based on the total weight of the preparations.

The embodiment particularly preferred according to the invention for thickening the surfactant systems according to the invention consists of the combination of ethoxylated glycerol isostearates with fatty alcohol polyglycol ethers. Particular preference according to the invention is given here to the combination of PEG-90 glyceryl isostearate with laureth-2 (e.g. Oxetal VD92, Zschimmer & Schwarz). It is particularly advantageous according to the invention to use ethoxylated glyceryl isostearates in a concentration of from 0.2 to 8% by weight and in particular from 1 to 2% by weight, based on the total weight of the formulation, and to use fatty alcohol polyglycol ethers in a concentration of from 0.1 to 5% by weight and in particular from 0.1 to 0.5% by weight, based on the total weight of the formulation.

The ratio of alkyl ether sulfates+alkylpolyamphopolycarboxyglycinates to ethoxylated glycerol isostearates is, in these thickened preparations, 1:1 to 20:1 and particularly preferably 8:1 to 15:1. In these thickened preparations, the ratio of sulfate and/or sulfonate surfactants to alkylpolyamphopolycarboxyglycinates is 10:1 to 2:1 and particularly preferably 3:1 to 6:1.

It is also advantageous to add complexing agents to the preparations according to the invention. The complexing agents are advantageously chosen from the group consisting of ethylenediaminetetraacetic acid (EDTA) and anions thereof, nitrilotriacetic acid (NTA) and anions thereof, hydroxyethylenediaminotriacetic acid (HOEDTA) and anions thereof, diethylene aminopentaacetic acid (DPTA) and anions thereof, trans-1,2-diaminocyclohexanetetraacetic acid (CDTA) and anions thereof, tetrasodium iminodisuccinate, and trisodium ethylenediamine disuccinate.

Apart from the abovementioned substances, the compositions comprise, in accordance with the invention, optionally the additives customary in cosmetics, for example perfume, dyes, antimicrobial substances, refatting agents, complexing and sequestering agents, pearlescent agents, plant extracts, vitamins, active ingredients, preservatives, bactericides, pigments which have a coloring action, thickeners, softening, moisturizing and/or humectant substances, or other customary constituents of a cosmetic or dermatological formulation, such as alcohols, polyols, polymers, foam stabilizers, electrolytes, organic solvents or silicone derivatives.

In a particular embodiment, the present invention relates to liquid soaps or washing lotions. Such products are used not only for washing the hands, but are usually also used for the entire body, including the face. Accordingly, they are also suitable for use as shower preparation. In developing these products, the dermatological requirements are at the forefront since the skin is in intensive contact with the concentrated surfactant solution. Particular emphasis is therefore placed on the choice of mild surfactants in low concentration. Further criteria are also a good foaming ability, and a pleasant, refreshing scent and the simultaneous care of the skin. Washing lotions and in particular shower baths usually have viscosities of from about 2 000 to 10 000 mPa·s which, on the one hand, permit good extensibility of the product with rapid foaming, but, on the other hand, should be sufficiently high to enable trouble-free application by hand or flannels.

Liquid soaps or washing lotions are generally characterized by a greater or lesser water content, but generally develop no noteworthy care effect since they have only a low oil content.

In addition, it is advantageous according to the invention to use the cosmetic cleansing formulations as bath, foam or shower bath formulation (e.g.: shower gels).

The examples below are intended to illustrate the present invention without limiting it. Unless stated otherwise, all amounts, proportions and percentages are based on the weight and the total amount or on the total weight of the preparations.

### EXAMPLES

| | 1 | 2 | 3 | 4 | 5 |
|---|---|---|---|---|---|
| Sodium laureth sulfate | 13.2% | 10% | 9.5% | 8% | 6% |
| Sodium | 1% | 1% | 2% | 2.5% | 1.5% |
| carboxymethylcocoylpolypropylamine | | | | | |
| Cocoamidopropylbetaine | 1.65% | 3.3% | — | — | 2% |
| Sodium cocoyl glutamate | 1.25% | 0.75% | 1.75% | 2% | 1.5% |
| PEG-40 hydrogenated castor oil | 0.50% | 0.50% | 0.50% | 0.50% | 0.50% |
| PEG-100 hydrogenated glyceryl palmitate | 0.50% | 0.50% | 0.50% | 0.50% | 0.50% |
| Polyquaterinium-10 | 0.05% | 0.1% | 0.2% | 0.1% | 0.15% |
| Sodium benzoate | 0.45% | 0.45% | 0.45% | 0.45% | 0.45% |
| Sodium salicylate | 0.20% | 0.20% | 0.20% | 0.20% | 0.20% |
| Citric acid | 0.50% | 0.50% | 0.50% | 0.50% | 0.50% |
| Perfume | q.s. | q.s. | q.s. | q.s. | q.s. |
| Water | ad | ad | ad | ad | ad |
| | 100 | 100 | 100 | 100 | 100 |

| | 6 | 7 | 8 | 9 |
|---|---|---|---|---|
| Sodium laureth sulfate | 10% | 12.8% | 14% | 10% |
| Sodium | 2.1% | 1.5% | 2% | 2% |
| carboxymethylcocoylpolypropylamine | | | | |
| Cocoamidopropylbetaine | 1.65% | 3.3% | — | — |
| Sodium cocoyl glutamate | 1.25% | 0.75% | 1.75% | 2% |
| PEG-90 glyceryl isostearate | 1.0% | 0.5% | 0.5% | 1.1% |
| Laureth-2 | 0.11% | 0.055% | 0.055% | 0.12% |
| PEG-40 hydrogenated castor oil | 0.50% | 0.50% | 0.50% | 0.6% |
| PEG-100 hydrogenated glyceryl palmitate | 0.50% | 0.50% | 0.50% | — |
| Polyquaterinium-10 | 0.05% | — | 0.2% | 0.2% |
| Sodium benzoate | 0.45% | 0.45% | 0.45% | 0.45% |
| Sodium salicylate | 0.20% | 0.20% | 0.40% | 0.4% |
| Citric acid | 0.50% | 0.50% | 0.50% | 0.9% |
| Benzophenone-4 | 0.05% | — | — | 0.05% |
| Styrene/acrylate/copolymer | — | 0.4% | — | — |
| EDTA | 0.2% | 0.2% | — | 0.2% |
| Perfume | q.s. | q.s. | q.s. | q.s. |
| Water | ad | ad 100 | ad 100 | ad |
| | 100 | | | 100 |

| | 10 | 11 | 12 |
|---|---|---|---|
| Sodium laureth sulfate | 8% | 9.5% | 12% |
| Sodium | 5% | 2% | 2% |
| carboxymethylcocoylpolypropylamine | | | |
| Cocoamidopropylbetaine | — | — | 2% |
| PEG-90 glyceryl isostearate | 3.4% | 1.7% | 1.7% |
| Laureth-2 | 0.4% | 0.2% | — |
| Sodium cocoyl glutamate | 1.75% | 2% | 1.5% |
| PEG-40 hydrogenated castor oil | 0.50% | 0.6% | 0.50% |
| PEG-100 hydrogenated glyceryl palmitate | 0.50% | — | 0.50% |
| Polyquaternium-10 | 0.2% | 0.2% | 0.15% |
| Sodium benzoate | 0.45% | 0.45% | 0.45% |
| Sodium salicylate | 0.20% | 0.4% | 0.20% |
| Citric acid | 0.50% | 0.9% | 0.50% |
| Benzophenone-4 | — | — | — |
| Styrene/acrylate copolymer | — | 0.4% | 0.4% |
| EDTA | 0.2% | 0.2% | 0.2% |
| Perfume | q.s. | q.s. | q.s. |
| Water | ad | ad | ad |
| | 100 | 100 | 100 |

That which is claimed:

1. A cosmetic cleansing formulation, comprising:
a) one or more surfactants selected from the group consisting of sulfates and sulfonates,
b) one or more alkylpolyamphopolycarboxyglycinates, and
c) one or more N-acylamino acid salts.

2. The cosmetic cleansing formulation as claimed in claim 1, wherein the one or more surfactants selected from the group consisting of sulfates and sulfonates include sodium laureth sulfate.

3. The cosmetic cleansing formulation as claimed in claim 2, wherein the sodium laureth sulfate is present in an amount between 0.1 and 30% by weight, based on the total weight of the formulation.

4. The cosmetic cleansing formulation as claimed in claim 2, wherein the sodium laureth sulfate is present in an amount between 9 and 15% by weight, based on the total weight of the formulation.

5. The cosmetic cleansing formulation as claimed in claim 2, wherein the sodium laureth sulfate is present in an amount of 10% by weight, based on the total weight of the formulation.

6. The cosmetic cleansing formulation as claimed in claim 2, wherein the one or more alkylpolyamphopolycarboxyglycinates include sodium carboxymethylco-coylpolypropylamine.

7. The cosmetic cleansing formulation as claimed in claim 2, wherein the one or more N-acylamino acid salts include sodium cocylglutamate.

8. The cosmetic cleansing formulation as claimed in claim 2, further comprising one or more ethoxylated glycerol isostearates in a concentration of from 0.2 to 8% by weight, based on the total weight of the formulation.

9. The cosmetic cleansing formulation as claimed in claim 2, further comprising one or more fatty alcohol polyglycol ethers in a concentration of from 0.1 to 5% by weight, based on the total weight of the formulation.

10. The cosmetic cleansing formulation as claimed in claim 1, wherein the one or more surfactants selected from the group consisting of sulfates and sulfonates are present in an amount between 0.1 and 30% by weight, based on the total weight of the formulation.

11. The cosmetic cleansing formulation as claimed in claim 1, wherein the one or more surfactants selected from the group consisting of sulfates and sulfonates are present in an amount between 9 and 15% by weight, based on the total weight of the formulation.

12. The cosmetic cleansing formulation as claimed in claim 1, wherein the one or more surfactants selected from the group consisting of sulfates and sulfonates are present in an amount of 10% by weight, based on the total weight of the formulation.

13. The cosmetic cleansing formulation as claimed in claim 1, wherein the one or more alkylpolyamphopolycarboxyglycinates include sodium carboxymethylcocoylpolypropylamine.

14. The cosmetic cleansing formulation as claimed in claim 1, wherein the one or more alkylpolyamphopolycarboxyglycinates are present in an amount between 0.1 and 10% by weight, based on the total weight of the formulation.

15. The cosmetic cleansing formulation as claimed in claim 1, wherein the one or more alkylpolyamphopolycarboxyglycinates are present in an amount between 0.1 and 5% by weight, based on the total weight of the formulation.

16. The cosmetic cleansing formulation as claimed in claim 1, wherein the one or more alkylpolyamphopolycarboxyglycinates are present in an amount of 2% by weight, based on the total weight of the formulation.

17. The cosmetic cleansing formulation as claimed in claim 1, wherein the one or more N-acylamino acid salts include sodium cocylglutamate.

18. The cosmetic cleansing formulation as claimed in claim 1, wherein the one or more N-acylamino acid salts are present in an amount between 0.1 and 10% by weight, based on the total weight of the formulation.

19. The cosmetic cleansing formulation as claimed in claim 1, wherein the one or more N-acylamino acid salts are present in an amount between 0.5 and 5% by weight, based on the total weight of the formulation.

20. The cosmetic cleansing formulation as claimed in claim 1, wherein the one or more N-acylamino acid salts are present in an amount of 2% by weight, based on the total weight of the formulation.

21. The cosmetic cleansing formulation as claimed in claim 1, further comprising one or more ethoxylated glycerol isostearates in a concentration of from 0.2 to 8% by weight, based on the total weight of the formulation.

22. The cosmetic cleansing formulation as claimed in claim 1, further comprising one or more fatty alcohol polyglycol ethers in a concentration of from 0.1 to 5% by weight, based on the total weight of the formulation.

23. A hair shampoo including the cosmetic cleansing formulation as claimed in claim 1.

24. A shower gel including the cosmetic cleansing formulation as claimed in claim 1.

25. A formulation for application to the skin, comprising:
sodium laureth sulfate;
sodium carboxymethylcocoylpolypropylamine; and
sodium cocylglutamate.

26. The formulation as claimed in claim 25, wherein the sodium laureth sulfate is present in an amount from 6 to 15% by weight, based on the total weight of the formulation.

27. The formulation as claimed in claim 26, wherein the sodium carboxymethylcocoylpolypropylamine is present in an amount from 1 to 5% by weight, based on the total weight of the formulation.

28. The formulation as claimed in claim 26, wherein the sodium cocylglutamate is present in an amount from 0.75 to 2% by weight, based on the total weight of the formulation.

29. The formulation as claimed in claim 25, further comprising one or more ethoxylated glycerol isostearates.

30. The formulation as claimed in claim 25, further comprising one or more fatty alcohol polyglycol ethers.

31. A method of cleansing the skin, comprising applying to the skin a formulation comprising:
a) one or more surfactants selected from the group consisting of sulfates and sulfonates,
b) one or more alkylpolyamphopolycarboxyglycinates, and
c) one or more N-acylamino acid salts.

32. The method as claimed in claim 31, wherein the one or more surfactants selected from the group consisting of sulfates and sulfonates include sodium laureth sulfate.
